(19) United States
(12) Patent Application Publication
(10) Pub. No.: US 2009/0018630 A1
(43) Pub. Date: Jan. 15, 2009

(54) SELF-EXPANDABLE EPIDURAL CORTICAL ELECTRODE

(76) Inventors: Thomas P. Osypka, Palm Harbor, FL (US); Hyoung-Ihl Kim, Kumku (KR); Yong-Il Shin, Iksan City (KR)

Correspondence Address: EDWARDS ANGELL PALMER & DODGE LLP, P.O. BOX 55874, BOSTON, MA, 02205, US

(21) Appl. No.: 12/172,160

(22) Filed: Jul. 11, 2008

Related U.S. Application Data
(60) Provisional application No. 60/955,859, filed on Aug. 14, 2007.

(30) Foreign Application Priority Data
Jul. 11, 2007 (KR) 10-2007-0069541

(51) Int. Cl. A61N 1/05

(52) U.S. Cl. 607/116

(57) ABSTRACT

A self-expandable epidural cortical electrode includes an electrically conductive expandable body and a connecting lead extending from the body. The body has an insulating layer on a first side and at least one region on a second side without an insulating layer. The connecting lead is adapted and configured for electrical communication with a control unit for providing power to the body electrical cortical stimulation.

### CROSS-REFERENCE TO RELATED APPLICATIONS

This application claims the benefit of priority to U.S. Provisional Application Ser. No. 60/955,859, filed Aug. 14, 2007, and to Korean patent application No. 10-2007-0069541, filed Jul. 11, 2007. Each of the foregoing applications is incorporated herein by reference in its entirety.

### FIELD OF THE INVENTION

The present invention relates to neurostimulation devices. More particularly, the present invention is directed to self-expandable cortical electrodes.

### BACKGROUND

Cortical stimulation of the brain is becoming an important tool to reactivate or to enhance the plasticity of the brain, in order to augment neurological recovery following brain injury.

A variety of brain electrodes are known in the art for applying to the brain cortex epidurally or intradurally. Of such devices, many are either too small to treat large regions of the brain, or require the relatively invasive step of opening a large portion of a patient's cranium, and accordingly may also require application of general anesthesia.

Moreover, coactivation of multiple regions of the brain is used to improve recovery following brain injury, such as following a stroke. Typically, to accomplish this one or more electrodes or electrode arrays covering a large area must be used. However, such multiple electrodes or electrode arrays may require multiple and/or large openings to be made in the cranium. Such openings may be considered relatively invasive by disturbing a relatively large portion of the cranium.

Thus, there remains a continued need in the art for a minimally invasive device capable of stimulating relatively large areas of a patient's brain. The present invention provides such a device and related methods and is a solution to the aforementioned problems.

### SUMMARY OF THE INVENTION

The purpose and advantages of the present invention will be set forth in and apparent from the description that follows.

The invention includes, in one aspect, a self-expandable epidural cortical electrode having an electrically conductive expandable body and a connecting lead extending from the body. The body has an insulating layer on a first side and at least one region on a second side without an insulating layer. The connecting lead is adapted and configured for electrical communication with a control unit for providing power to the body electrical cortical stimulation. The self-expandable epidural cortical electrode can further include a sheath for covering the body that, when covering the body, holds the body in a collapsed or otherwise compressed state.

The self-expandable epidural cortical electrode can further include one or more control wires adapted and configured to deploy and retract the electrically conductive expandable body. The electrically conductive expandable body can be formed by an open framework of material defining internal apertures therein. Discrete conductive regions can be exposed on the electrically conductive expandable body, and adapted and configured to be in electrical communication with a target tissue. The electrically conductive expandable body can include hinge regions defined thereon to facilitate compression of the expandable body into a compressed state.

In accordance with another aspect of the invention, a self-expandable epidural cortical electrode includes an expandable body having one or more electrodes defined thereon and a connecting lead extending from the body, adapted and configured for electrical communication with a control unit. The expandable body can be formed of a conductive material, which material can be a shape-memory alloy.

Alternatively, the expandable body can be formed of a non-conductive material, and the expandable body can include electrically-conductive electrodes and conduction paths applied thereto. An insulating material can be selectively arranged on the expandable body to allow selected regions to be in electrical communication with an external target tissue.

In accordance with another aspect of the invention, a method of inserting a self-expandable epidural cortical electrode includes the steps of forming an aperture in a patient's cranium, inserting an electrode body in a compressed state through the aperture, and deploying the electrode body into an expanded state.

The step of deploying the electrode body can include the step of removing a sheath provided on the electrode body to maintain the body in a compressed state. The method can further include withdrawing the sheath through the aperture formed in the patient's cranium.

It is to be understood that both the foregoing general description and the following detailed description are exemplary and are intended to provide further explanation of the invention.

### BRIEF DESCRIPTION OF THE DRAWINGS

The accompanying drawings, which are incorporated in and constitute part of this specification, are included to illustrate and provide a further understanding of the invention. Together with the description, the drawings serve to explain the principles of the invention, wherein:

FIG. 1 is an isometric view of a self-expandable epidural cortical electrode assembly in accordance with the invention, illustrating the electrode in a compressed state;

FIG. 2 is a cross-section of the self-expandable epidural cortical electrode assembly of FIG. 1, taken along line A-A′;

FIG. 3 is an isometric view of the self-expandable epidural cortical electrode assembly of FIG. 1, illustrating the electrode in an expanded state;

FIG. 4 is an isometric view of the self-expandable epidural cortical electrode assembly of FIG. 1, illustrating the electrode in a partially expanded state;

FIG. 5A illustrates an initial insertion step of a self-expandable epidural cortical electrode assembly into the cranium of a patient, in accordance with the invention;

FIG. 5B illustrates a deployment step of a self-expandable epidural cortical electrode assembly into the cranium of a patient, in accordance with the invention;

FIG. 5C illustrates a self-expandable epidural cortical electrode assembly in accordance with the invention in a fully deployed state, with a sheath of the assembly being removed from within the cranium;

FIG. 6 illustrates an alternate mechanism for compressing an epidural cortical electrode assembly in accordance with the invention;

FIG. 7 illustrates an epidural cortical electrode assembly in accordance with the invention in an open state arranged on one example region of a brain cortex; and

FIGS. 8A-8C illustrate an epidural cortical electrode assembly in accordance with the invention, in an open state arranged in different example regions of a brain cortex.

### DETAILED DESCRIPTION OF THE PREFERRED EMBODIMENT

Reference will now be made in detail to example embodiments of the invention, which are illustrated in the accompanying drawings. The methods of the invention will be described in conjunction with the detailed description of the respective devices.

The devices and methods presented herein may be used for inhibitory cortical stimulation of the brain for any of a number of brain disorders, including epilepsy or to enhance recovery following brain injury, for example, stroke or head injury. The present invention provides a minimally invasive device capable of stimulating a relatively large area of the brain cortex without necessitating forming a large opening in the patient's cranium, which may only require local anesthesia. Further, electrodes in accordance with the invention are easily removed from the patient, without necessitating re-opening a large portion of the patient's cranium.

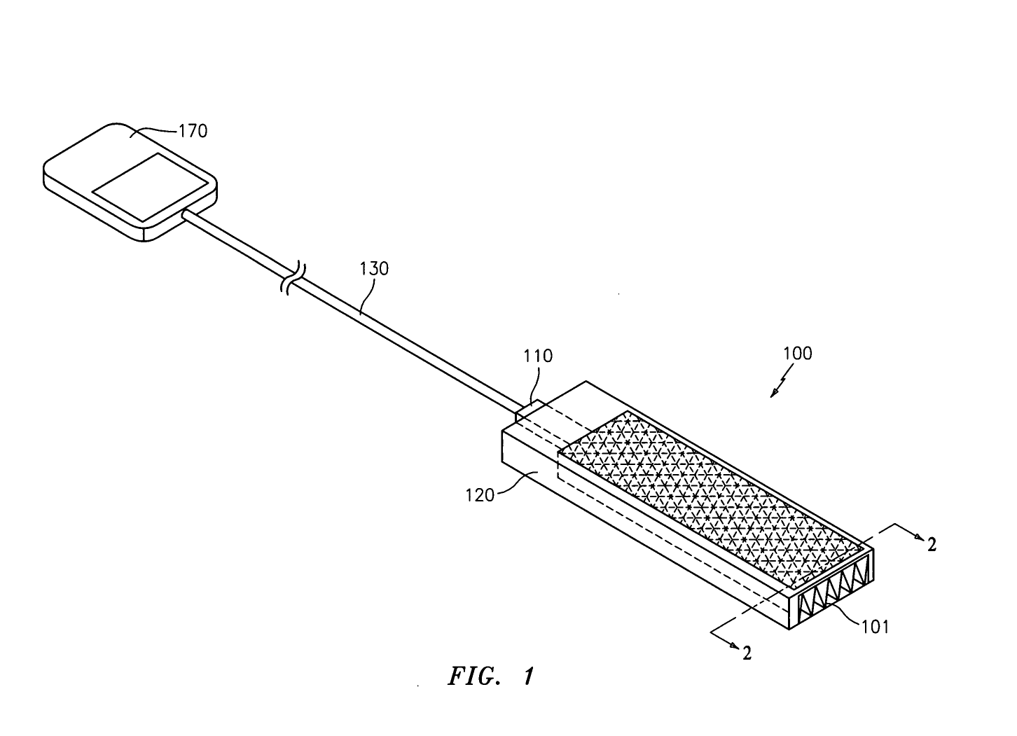

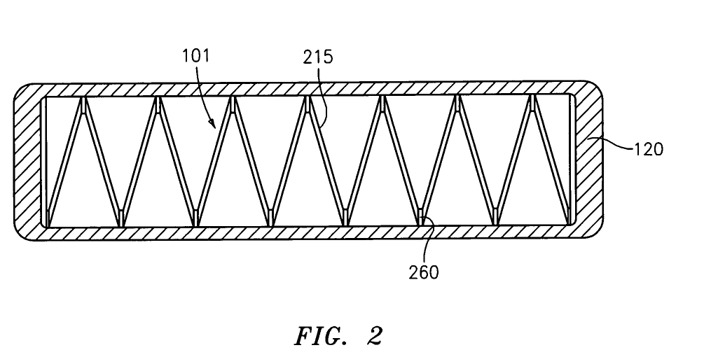

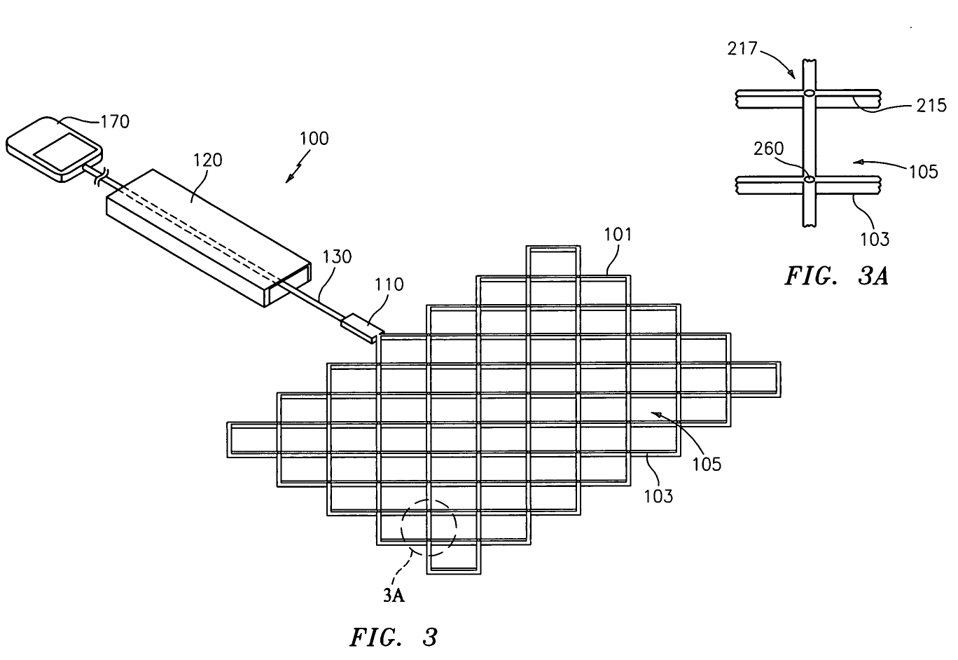

FIG. 1 illustrates a self-expandable epidural cortical electrode assembly 100 in accordance with the invention in a collapsed state, while FIG. 2 is a cross-sectional view of the assembly 100, taken across like A-A′. FIG. 3 illustrates the electrode assembly 100, with the electrode 101 in a deployed state. As illustrated, and best seen in FIG. 3, the electrode 101 includes a conductive framework 103, with open regions 105 defined within the framework 103. The electrode 101 can be formed of a collapsible or compressible structure, such as, for example, a wire net of any suitable configuration, or a structure similar to those used in cardiovascular stents, as know in the art. FIG. 1 further illustrates a control unit 170 connected by way of a connecting lead 130 to the electrode 101 by way of a connecting element 110.

The electrode 101 is preferably formed of a conductive material, such as, but not limited to, a titanium alloy or stainless steel, and may be formed of a shape-memory alloy such as a nickel-titanium alloy. An insulating layer is preferably provided on the electrode 101 on at least a first side thereof, which side is intended to contact anatomy opposite the brain cortex of a patient. If the electrode 101 is applied epidurally, the insulated first side will contact the inner surface of the patient's cranium, with the opposite, second side of the electrode 101 resting against the dura of the brain. If the electrode 101 is applied intradurally, the insulated first side will contact the inner surface of the dura of the brain, with the second side of the electrode 101 resting against the cortex. The second side of the electrode 101 can include insulated areas, if so desired. Alternatively, the second side of the electrode 101 can include multiple areas of insulation, and multiple areas free of insulation, so as to apply therapeutic stimulation to discrete areas. Alternatively still, the second side can be insulated, and separate electrodes can be provided on the external surface of the insulated second side to deliver therapeutic stimulation to discrete areas. Any suitable type of biocompatible, flexible and electrically insulating material can be used, such as silicone, for example.

In accordance with a preferred aspect of the invention, the electrode 101 deploys laterally, in an essentially 2-dimensional manner, so as to exert a minimal amount of pressure on the dura and brain of the patient, so as to reduce the potential for brain injury. Accordingly, the subject electrode is relatively thin. In accordance with one embodiment, the electrode 101 is between about 0.2 and 1.0 mm in thickness. If necessary, however, the electrode can be thinner or thicker.

Alternatively, the electrode 101 itself can be formed of a non-conductive material, such as a resilient biocompatible polymeric or composite material, and can include discrete electrodes provided thereon. Such electrodes can be connected to an external controller by way of discrete conduction paths in the form of wires, or alternatively by conduction paths printed or otherwise applied to the polymeric body. In this manner, an electrode pattern can be applied to the body 101, in any manner desirable. Additionally, multiple separate electrically conductive circuits can simply be added, as desired, without significantly increasing the bulk of the electrode body.

The desired electrode pattern can alternatively be achieved by selectively applying an insulating layer over a body. Such body can either a conductive body or, alternatively, as set forth above, a non-conductive body having conduction paths provided thereon.

In accordance with one aspect, and as best seen in FIGS. 2 and 3, an insulating layer 215 covers a large portion of the electrode body 101, and electrode regions 260 are left without insulation, so as to be in electrical communication with the tissue, against which the electrode body 101 is placed. Such regions 260 can be provided in any portion of the electrode body 101, but in the illustrated embodiment are provided at the joints 217 at the intersection of the framework elements 103, and can be embodied to function as hinges, as described in more detail below.

The self-expandable epidural cortical electrode assembly 100 can be adapted and configured to collapse, fold or roll, in order be in a more compact form for insertion through an aperture formed in a patient's cranium. The joints 217 can include any structural adaptation necessary to function as hinges, in order to fold, collapse or bend appropriately. Such adaptations can include, for example, weakened regions, pivots, such as by providing a rotatable engagement or pin therebetween, or the like. Alternatively, the joints 217 can be formed by welding or otherwise fusing overlapping portions of the electrode 101. The joints 217 are preferably configured so as to allow the electrode 101 to be collapsed, bent or otherwise compressed along its length during insertion or removal through an aperture formed in the cranium.

FIG. 1 illustrates the electrode assembly 100, with the electrode 101 in a compact state, enclosed in a sheath 120. The sheath 120 can either fully or partially cover the electrode 101, depending on the precise implementation. When withdrawn, as illustrated in FIG. 3, the sheath 120 allows the electrode 101 to expand. The sheath 120 can simply slide proximally from the electrode 101, and be pulled through the cranium and removed. Alternatively, the sheath 120 can include a rupturable portion or a cord that causes unraveling of a seam formed in the sheath 120, to allow the sheath 120 to be more easily removed from the electrode assembly 100.

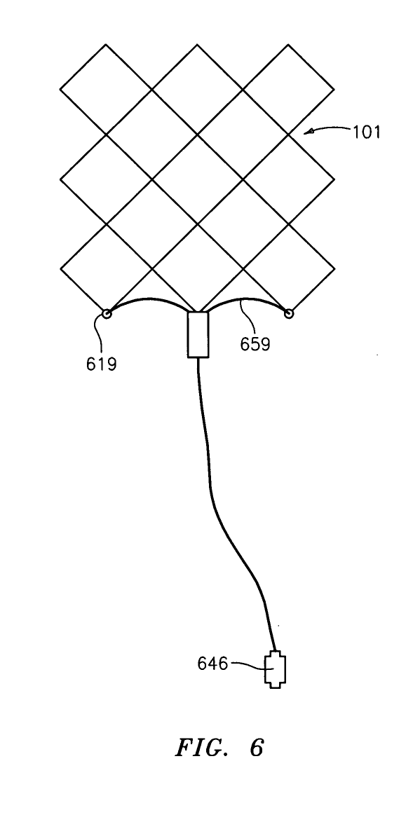

FIG. 3 also illustrates insulation 215 carried by the electrode 101, and a connecting lead 130 for connecting the electrode 101 to a power source, such as an implantable control unit or pulse generator 170. The connecting lead 130 can be adapted and configured to terminate at its distal end in a connecting terminal (e.g. 640 in FIG. 6) for interfacing directly with the controller or other power source. An intermediate connection element 110 may be provided on the electrode 101 to secure the electrode 101 to the connecting lead 130. The connection element can simply be a reinforcement to reinforce an electrical connection between the electrode 101 and the connecting lead 130, which may be mutually welded, soldered or connected by other methods.

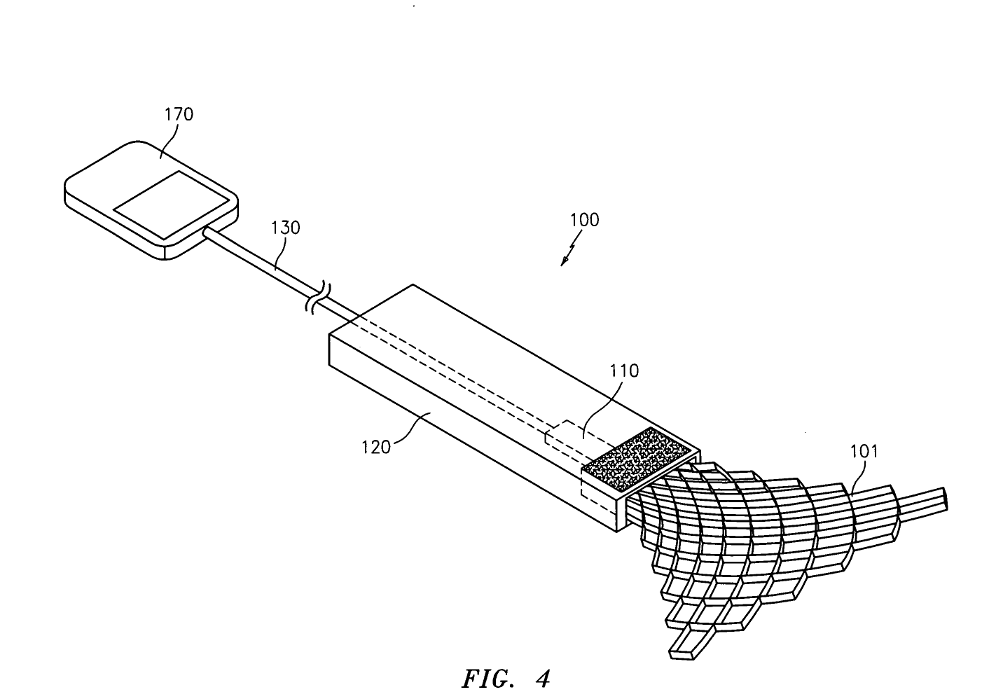

FIG. 4 illustrates the electrode assembly 100, with the electrode 101 expanding as the sheath 120 is withdrawn therefrom.

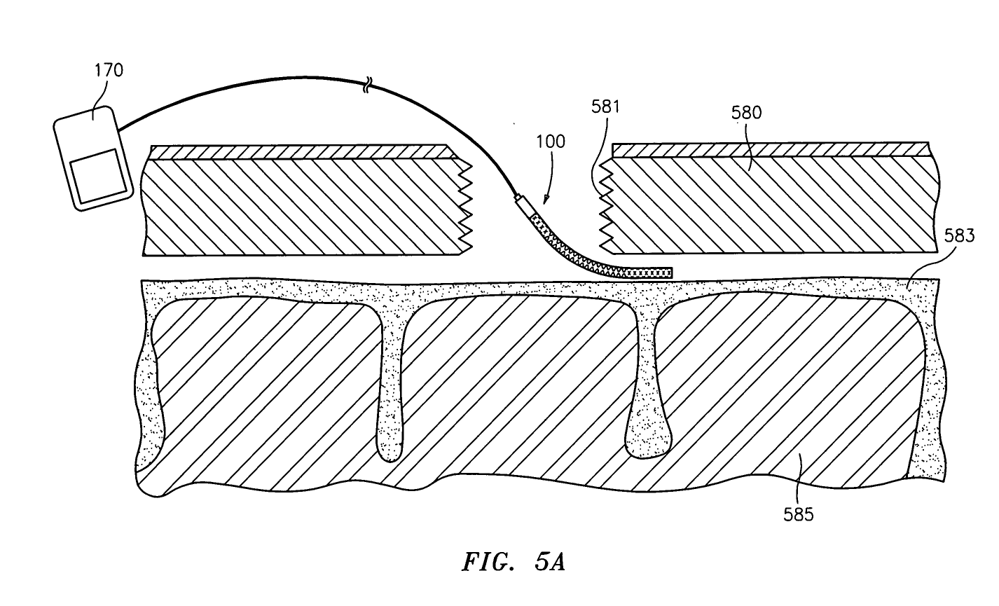

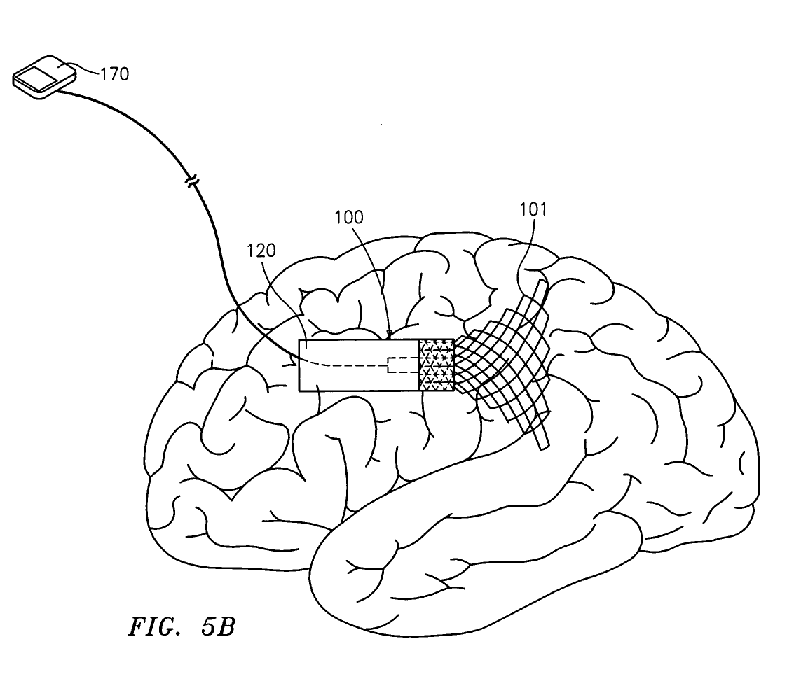

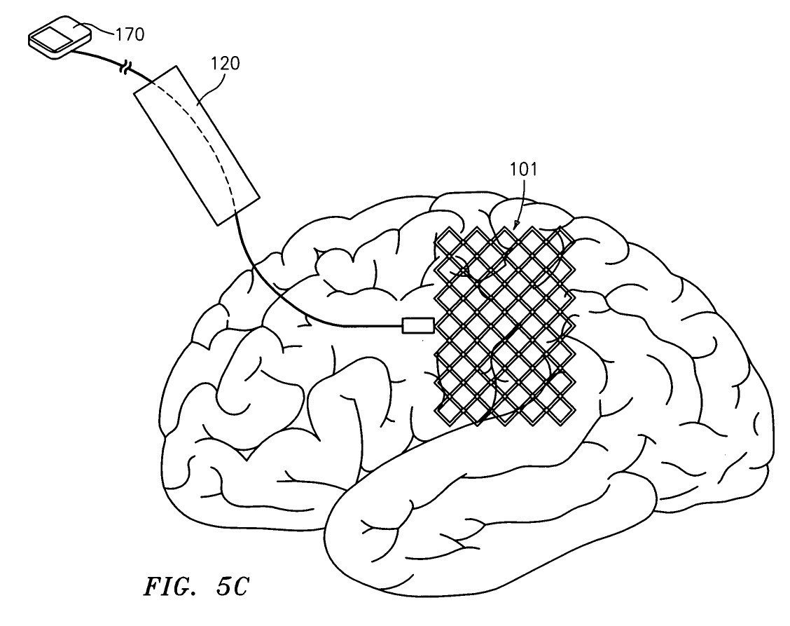

FIGS. 5A-5B illustrate example steps involved in implanting the subject electrode assembly 100. First, an aperture 581 is formed in the cranium 580. The electrode 101 and sheath 120 of the assembly 100 are inserted through the aperture 581 in the compressed state, as best illustrated in FIG. 5A. In the illustrated embodiment, the assembly is placed between the cranium 580 and the dura 583 of the brain 585. The electrode assembly is placed in the desired location, and the sheath is withdrawn, which allows the electrode 101 to expand, as illustrated in FIG. 5B. The sheath 120 is withdrawn through the aperture 581 formed in the cranium 580, as illustrated in FIG. 5C. Withdrawal of the sheath 120 can be effected by way of a guide rod, cable or the like. Subsequently, the aperture 581 can be covered, if necessary, an incision can be closed. The power source 170 can be implanted in the patient, as would be a cardiac pacemaker, for example. Alternatively, the connecting lead 130 can terminate external to the patient's body, if short term treatment is desired. The electrode 100 can be embodied such that the electrode 101 can be re-compressed and removed from the patient by re-applying the sheath 120 in the reverse order from the procedure for expanding the electrode 100, illustrated in FIGS. 5A-5C.

In an alternate embodiment, as illustrated in FIG. 6, connection points, which may be embodied as holes 619 can be provided on the proximal edge of the electrode 101, to which one or more control wires 659 or the like are connected. The wire(s) 659 can be embodied so as to deploy the electrode 101 following insertion, and/or to retract the electrode 101 prior to removal. During use of the electrode 101, the wire(s) 659 can be anchored to the cranium of the patient, which allows for anchoring of the electrode 100. Such an arrangement facilitates a surgeon in easily locating the proximal end(s) of the wire(s) 459 to initiate removal of the electrode 101. For removal of the electrode 101, the wire(s) 659 are pulled. The electrode 101 then collapses and can be easily introduced into a sheath or other case. The electrode 101 can be embodied such that once the proximal end of electrode 101 is in the sheath, the remaining part of the electrode 101 can automatically collapse while the electrode 101 is pulled. Additionally, if so desired, the cables 659 can be electrically conductive, and can connect with additional stimulation circuits provided within the electrode body 101. Such circuits can be defined by separate conduction elements, mutually electrically isolated but physically connected by insulating elements, to form the electrode body 101.

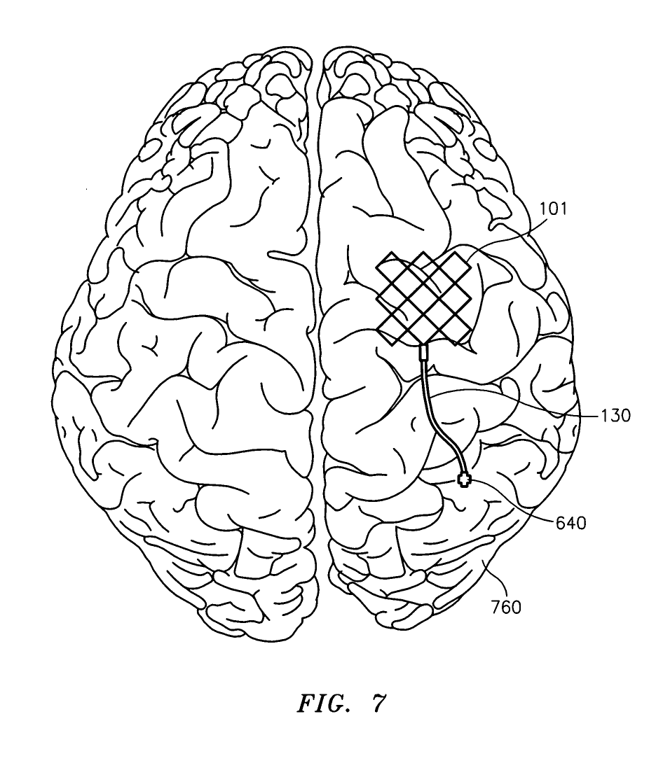

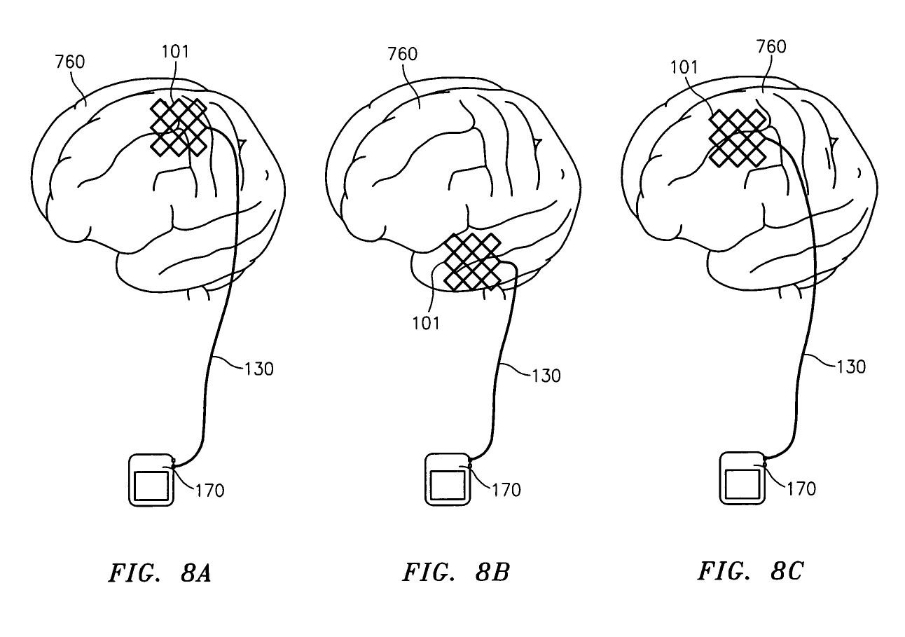

FIGS. 7 and 8A-8C show the electrode 101 in accordance with the invention in an expanded state in selected locations with respect to a brain 760. FIG. 7 illustrates the electrode 101 arranged on the upper region of the brain 760 in the premotor and motor cortex areas for treatment of chronic central pain or chronic stroke. FIG. 8A illustrates the electrode 101 arranged on the premotor and motor cortex areas of the brain 760 for treatment of chronic pain or stroke, FIG. 8B illustrates the electrode 101 arranged on the temporal area of the brain 760 for treatment of temporal lobe epilepsy, and FIG. 8C illustrates the electrode 101 arranged on the prefrontal area of the brain 760 for treatment of depression or Alzheimer's disease, for example. In each situation, the electrode 101 is connected by way of the connecting lead 130 to a control unit 170. The control unit 170 can be, for example a pulse generator and can be arranged in a convenient location as, for example, a pace-maker style control unit placed in the chest wall of the patient. Alternatively, a low-profile generator placed in the cranium can be used. Alternatively still, the connecting terminal 640 can be connected to an induction coil that receives power through induction from an external device.

If desired, distinctive markings can be provided on electrode 101, so that the first or upper surface of the electrode 101 and second or lower surface of the electrode 101 can be easily distinguished.

The devices and methods of the present invention, as described above and shown in the drawings, provide for a electrodes with superior properties. It will be apparent to those skilled in the art that various modifications and variations can be made in the device and method of the present invention without departing from the spirit or scope of the invention. Thus, it is intended that the present invention include such modifications and variations.

What is claimed is:

1. A self-expandable epidural cortical electrode comprising:
(a) an electrically conductive expandable body having an insulating layer on a first side and at least one region on a second side without an insulating layer; and
(b) a connecting lead extending from the body, adapted and configured for electrical communication with a control unit for providing power to the body.

2. The self-expandable epidural cortical electrode of claim 1, further comprising a sheath for covering the body that, when covering the body, holds the body in a compressed state.

3. The self-expandable epidural cortical electrode of claim 1, further comprising one or more control wires adapted and configured to deploy and retract the electrically conductive expandable body.

4. The expandable epidural cortical electrode of claim 1, wherein the electrically conductive expandable body is formed by an open framework of material defining internal apertures therein.

5. The expandable epidural cortical electrode of claim 1, wherein discrete conductive regions are exposed on the electrically conductive expandable body, and adapted and configured to be in electrical communication with a target tissue.

6. The expandable epidural cortical electrode of claim 1, wherein the electrically conductive expandable body includes hinge regions defined thereon to facilitate compression of the expandable body into a compressed state.

7. A self-expandable epidural cortical electrode comprising:
(a) an expandable body having one or more electrodes defined thereon; and
(b) a connecting lead extending from the body, adapted and configured for electrical communication with a control unit.

8. The self-expandable epidural cortical electrode of claim 7, wherein the expandable body is formed of a conductive material.

9. The self-expandable epidural cortical electrode of claim 8, wherein the conductive material is a shape-memory alloy.

10. The self-expandable epidural cortical electrode of claim 7, wherein the expandable body is formed of a non-conductive material.

11. The self-expandable epidural cortical electrode of claim 10, wherein the expandable body includes electrically-conductive electrodes and conduction paths applied thereto.

12. The self-expandable epidural cortical electrode of claim 7, wherein an insulating material is selectively arranged on the expandable body to allow selected regions to be in electrical communication with an external target tissue.

13. A method of inserting a self-expandable epidural cortical electrode comprising:
(a) forming an aperture in a patient's cranium;
(b) inserting an electrode body in a compressed state through the aperture; and
(c) deploying the electrode body into an expanded state.

14. The method of claim 13, wherein the step of deploying the electrode body comprises the step of removing a sheath provided on the electrode body to maintain the body in a compressed state.

15. The method of claim 14, wherein the method further includes withdrawing the sheath through the aperture formed in the patient's cranium.
